(19) United States
(12) Patent Application Publication
(10) Pub. No.: US 2003/0161798 A1
(43) Pub. Date: Aug. 28, 2003

(54) Cosmetic stick compositions with polyolefin elastomers

(75) Inventors: David Martin Kellner, Hollis, NY (US); Christopher James Cashin, Dunellen, NJ (US); Anjali Abhimanyu Patil, Westfield, NY (US)

Correspondence Address: Julie Blackburn, Esq., Revlon Consumer Products Corporation, Law Dept., 625 Madison Avenue, New York, NY, 10022, US

(73) Assignee: Revlon Consumer Products Corporation, New York, NY

(21) Appl. No.: 10/383,341

(22) Filed: Mar. 10, 2003

Related U.S. Application Data
(63) Continuation of application No. 09/277,157, filed on Mar. 26, 1999.
Application No. 09/277,157 is a continuation of application No. 08/730,955, filed on Oct. 16, 1996, now abandoned.

(51) Int. Cl. A61K 7/025

(52) U.S. Cl. 424/64

(57) ABSTRACT

A cosmetic stick composition comprising, by weight of the total composition:

0.1-10% of a polyolefin elastomer,

10-70% of a solubilizer for the polyolefin elastomer, and

5-60% of a wax-type solidifying agent, and

### TECHNICAL FIELD

The invention is in the field of cosmetic compositions in the form of solid sticks such as antiperspirants, deodorants, sun care, moisturizing, or analgesic sticks as well as lipsticks.

### BACKGROUND OF THE INVENTION

Cosmetic stick compositions are widely used for cosmetic and personal care purposes, such as antiperspirants, deodorants, sun-blocks, lipsticks, and the like. There are basically three types of cosmetic sticks: compressed powder sticks, gel sticks, and wax sticks. Compressed powder sticks are often quite brittle and hard and tend to leave a residue on the skin. Gel sticks may leave a wet, greasy residue on application. Wax sticks may also exhibit problems with stickiness, greasiness, and hardness. In addition, they exhibit the additional problem of syneresis, which is the separation of components within the stick.

There is a need for cosmetic sticks which have an acceptable hardness, which do not provide a greasy, sticky residue, and which are easy to use and feel comfortable on application, and, in addition, exhibit minimal syneresis.

### SUMMARY OF THE INVENTION

The invention is directed to a cosmetic stick compositions comprising, by weight of the total composition:

0.1-10% of a polyolefin elastomer,

10-70% of a solubilizer for the polyolefin elastomer, and

5-60% of a wax-type solidifying agent.

### DETAILED DESCRIPTION

All percentages mentioned herein are percentages by weight unless otherwise indicated. Preferably the cosmetic sticks of the invention are “anhydrous”. The term “anhydrous” means that the stick contains less than about 5% by weight water, or in other words, water is not intentially added to the compositions. The cosmetic sticks of the invention may also be translucent, although they can be formulated in accordance with the invention so that they are not translucent. The compositions of the invention contain the following ingredients:

### The Polyolefin Elastomer

The composition of the invention contains 0.1-10%, preferably 0.5-8%, more preferably 1-7% of polyolefin elastomer. Suitable polyolefin elastomers are disclosed in U.S. Pat. Nos. 5,272,236 and 5,278,272, which are hereby incorporated by reference. In particular, the polyolefin elastomers are substantially linear and have a density of 0.85 to 0.97 grams per cubic centimeter. The elastomers can be homopolymers of C₂₋₂₀ alpha-olefins, such as ethylene, propylene, 4-methyl-1-pentene, etc., or they can be copolymers of ethylene with at least one C₃₋₂₀ alpha-olefin and/or C₂₋₂₀ acetylenically unsaturated monomers, and/or C₄₋₁₈ diolefins. The polymers of the invention can also be copolymers of ethylene with at least one of the above C₃₋₂₀ alpha-olefins, diolefins, and/or acetylenically unsaturated monomers in combination with other unsaturated monomers. Preferably, the polyolefin elastomer is a copolymer of ethylene and a C₃₋₂₀ alpha-olefin. Most preferably, the polyolefin elastomer has the general formula:

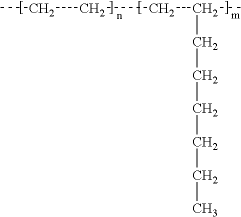

wherein the number of m and n repeating units are sufficient to provide an elastomer having a melting point of about 50 to 70° C. Preferably, there are approximately about 3 repeating ethylene units for every alpha olefin unit, or in other words the ratio of n to m is about 3 to 1. Most preferably, m and n are each independently 1-1,000,000. Polyolefin elastomers having these characteristics can be purchased from Dow Chemical Company and are sold under the tradename ENGAGE POE.

### The Solubilizer for the Polyolefin Elastomer

The composition comprises 10-70%, preferably 12-60%, more preferably 15-55% of a solubilizer for the polyolefin elastomer, or “POE solubilizer”. The term “POE solubilizer” means an ingredient which is capable of solubilizing the POE, which is a tough, flexible thermoplastic material which, alone, is not generally not compatible with the ingredients which are usually found in cosmetic sticks. In particular, the POE solubilizer must be such that the POE must be soluble in, or capable of dissolving in, the POE solubilizer when the POE is heated to approximately 150 to 200° C. and mixed with POE solubilizer. At the same time the POE solubilizer must be compatible with the other ingredients in the formula such as the wax-type solidifying agent and other optional ingredients such as the antiperspirant or deodorant salt, the emulsifier, etc. The POE solublizer is a liquid at room temperature and may be volatile or non-volatile. The term “volatile” means that the POE solubilizer has a vapor pressure of at least 2 mm. of mercury at 20° C. The term “non-volatile” means that the POE solubilizer has a vapor pressure of less than 2 mm. of mercury at 20° C.

Examples of POE solubilizers include straight or branched chain paraffinic hydrocarbon fluids having 5 to 50, preferably 8 to 20 carbons in the alkyl chain. Examples of such paraffinic hydrocarbons are disclosed in U.S. Pat. Nos. 4,724,139, 3,439,088 and 3,818,105, all of which are hereby incorporated by reference. Suitable paraffinic hydrocarbons are sold by Exxon under the Isopars tradename, or Permethyl Corporation under the Permethyl tradenames. In general, these hydrocarbons have an average molecular weight of from about 70 to 190, a boiling point of about 30-320° C., a viscosity of 0.5 to 25 centistokes at 25° C. Preferably the POE solubilizer is a non-volatile paraffinic hydrocarbon fluid, in particular a branched chain aliphatic hydrocarbon having 8 to 50 carbons in the alkyl chain. Examples of POE solubilizers are the straight or branched chain forms of decane, dodecane, tetradecane, tridecane, tetradecane, pentadecane, hexadecane, heptadecane, octadecane, nonadecane, eicosane, eicosane, heneicosane, docosane, docosane, tricosane, tetracosane, pentacosane, and the like, as well as C₇₋₈ isoparaffins, C₈₋₉ isoparaffin, C₉₋₁₁ isoparaffin, C₉₋₁₃ isoparaffin, C₉₋₁₄ isoparaffin, C₁₀₋₁₁ isoparaffin, C₁₀₋₁₃ isoparaffin, C₁₁₋₁₂ isoparaffin, C₁₁₋₁₃ isoparaffin, C₁₂₋₁₄ isoparaffin, C₁₃₋₁₄ isoparaffin, C₁₃₋₁₆ isoparaffin, C₂₀₋₄₀ isoparaffin, hydrogenated polyisobutene, and so on. Most preferably, the POE solubilizer is volatile, and is isoeicosane.

### The Wax-Type Solidifying Agent

The compositions of the invention contain 5-60%, preferably 7-50%, more preferably 8-45% of a wax-type solidifying agent. The wax-type solidifying agent is a solid at room temperature and generally has a melting point of 35 to 120° C. The solidifying agent may be a a high melting point wax, a low melting point wax, or mixtures thereof. High melting point waxes generally have a melting point of 65 to 120° C. Examples of such high melting point waxes include beeswax, spermaceti, carnauba, bayberry, candelilla, montan, ozokerite, ceresin, paraffin, synthetic waxes such as polyethylene or Fisher-Tropsch waxes, microcrystalline, and mixtures of these waxes. The low melting point waxes generally have a melting point of 35 to 65° C. Examples of low melting point waxes include fatty acids, fatty alcohols, fatty acid esters, fatty acid amides having chains from about 8 to 30 carbons, and mixtures thereof. Examples of such low melting point waxes include cetyl alcohol, stearyl alcohol, palmitic acid, behenamide, sucrose esters of tallow fatty acids, mono and di-fatty acid esters of polyethylene glycol, silicone waxes such as stearyl dimethicone, stearoxy dimethicone, cetyl dimethicone, behenyl dimethicone, dimethicone behenate, and mixtures thereof.

In the preferred embodiment of the invention, the composition contains 5-60% of high melting point waxes. Most preferably, the high melting point waxes are straight or branched chain hydrocarbons such as paraffin, polyethylene, microcrystalline, and the like. The preferred waxes are microcrystalline, paraffin, and polethylene.

### Other Ingredients

The cosmetic stick composition may comprise a variety of additional ingredients as follows:

### Antiperspirant Actives

In the case where the stick serves as a base for an antiperspirant, the stick comprises 1-30% of an antiperspirant active salt, thus the sticks can be referred to suspensoid sticks because the antiperspirant salts will be in the particulate, suspended form in the stick. The term “antiperspirant salt” means any compound or composition having antiperspirant activity, preferably astringent metallic salts such as the inorganic and organic salts of aluminum, zirconium, and zinc, and mixtures thereof. Particularly preferred are the aluminum and zirconium salts such as aluminum halides, aluminum hydroxide halides, zirconyl oxide halides, zirconyl hydroxy halides, and mixtures thereof. Aluminum salts include those of the formula:

Al₂(OH)ₐCl_{b}.xH2O

wherein a is from about 2 to 5; a+b=6; x is from about 1 to about 6; and wherein a, b, and x may have non-integer values. Zirconium salts include those of the formula:

ZrO(OH)₂₋ₐClₐ.xH₂O

wherein a is from about 1.5 to about 1.87; x is from about 1 to about 7; and wherein a and n may have non-integer values.

Examples of aluminum and zirconium salts include aluminum chloride, aluminum chlorohydrate, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, aluminum zirconium octachlorohdrate, aluminum zirconium octachloroydrex GLY, aluminum zirconium pentachlorohydrate, aluminum zirconium pentachlorohydrex GLY, aluminum zirconium tetrachlorohydrate, aluminum zirconium tetrachlorohydrex GLY, aluminum zirconium trichlorohydrate, aluminum zirconium trichlorohydrex GLY, and mixtures thereof.

Particularly preferred zirconium salts are those complexes also containing aluminum and glycine, in particular, aluminum zirconium tetrachlorohydrex GLY.

### Deodorant Actives

In the case where the cosmetic stick functions as a deodorant, the stick may contain 0.1-30% of a deodorant active. Examples of suitable deodorant actives include fragrances, ammonium phenolsulfonate, benzalkonium chloride, benzethonium chloride, bromochlorophene, cetylpyridinium chloride, chlorophyllin-copper complex, chlorothymol, chloroxylenol, cloflucarban, dequalinium chloride, dichlorophene, dichloro-m-xylenol, disodium dihydroxyethyl sulfosuccinylundecylenate, domiphen bromide, hexachlorophene, lauryl pyridinium chloride, methylbenzethonium chloride, phenol, sodium bicarbonate, sodium phenolsulfonate, triclocarbone, triclosan, zinc phenolsulfonate, zinc ricinoleate, and mixtures thereof. The preferred deodorant active is triclosan or fragrance.

### Emollient

The cosmetic stick compositions of the invention preferably contain 1-60%, preferably 5-50%, more preferably 8-45% of an emollient oil. Suitable emollients are liquids at room temperature, and preferably insoluble in water. Examples of emollients include water insoluble non-volatile silicones such as polyalkylsiloxanes, polyarylsiloxanes, and polyethersiloxane copolymers having a viscosity of 4 to 100,000 centistokes at 25° C. Suitable silicones having these characeristics can be purchased from Dow Corning Corporation under the tradenames Dow Corning 200 series fluids, 344 fluid, 345 fluid; Union Carbide Corporation under the Silicone 7207 and Silicone 7158 tradenames; and General Electric under the SF 1075 tradename.

The emollient may also comprise esters (other than alkoxylated carboxylic acids), particularly fatty acid and/or fatty alcohol esters. These esters exhibit the general formula RCO-OR′ wherein RCO is the carboxylic acid radical and OR′ is the alcohol residue. Examples of such esters include diisopropyl sebacate, diisopropyl adipate, isopropyl myristate, isopropyl palmitate, ethyl hexyl palmitate, behenyl behenate, behenyl erucate, dibutyl sebacate, dihexyl adipate, diisobutyl adipate, dimethicone copolyol acetate, dioctyl malate, dioctyl succinate, disteareth-2 lauroyl glutamate, and so on. Preferred is an ester which is the reaction product of sebacic acid and isopropyl alcohol, namely diisopropyl sebacate.

Glyceryl esters, preferably fatty acid mono- and diglycerides or triglycerides modified by reaction with alcohols, also are suitable emollients. Examples of such glyceryl esters include glyceryl stearate, glyceryl myristate, glyceryl dioleate, glyceryl erucate, glyceryl dipalmitate, hydrogenated coco-glycerides, glyceryl linoleate, glyceryl laurate, and mixtures thereof.

Lanolin and lanolin derivatives are also suitable emollient oils, as well as mineral oil, and the like.

### The Emulsifier

The compositions of the invention preferably contain 0.001-5%, preferably 0.01-4%, more preferably 0.1-3% of an emulsifier. The emulsifier aids in “wash off”, i.e. it makes the stick composition easier to wash off the skin. In addition, the emulsifier aids in providing a more translucent stick. Suitable emulsifiers include alkoxylated alcohols or alkoxylated carboxylic acids. Alkoxylated alcohols are ethers formed from the reaction of an alcohol with an alkylene oxide, generally ethylene oxide or propylene oxide. Examples of alkoxylated alcohols are beheneth 5-30 (the 5-30 meaning the number of repeating ethylene oxide or propylene oxide units), Ceteareth 2-100, Ceteth 1-45, Cetoleth 24-25, Choleth 10-24, Coceth 3-10, C9-11 pareth 3-8, C11-15 pareth 5-40, C11-21 Pareth 3-10, C12-13 pareth 3-15, Deceth 4-6, Dodoxynol 5-12, Glycereth 7-26, Isoceteth 10-30, Isodeceth 4-6, Isolaureth 3-6, isosteareth 3-50, Laneth 5-75, Laureth 1-40, Nonoxynol 1-120, Nony nonoxynol 5-150, Octoxynol 3-70, Oleth 2-50, PEG 4-350, Steareth 2-100, Trideceth 2-10, and so on. Preferred is Steareth-100.

Alkoxylated carboxylic acids are formed when a carboxylic acid is reacted with an alkylene oxide or with a pre-formed polymeric ether. The resulting product may be a monoester, diester, or mixture thereof. Examples of such alkoxylated carboxylic acids in PEG-8 distearate, PEG-8 behenate, PEG-8 caprate, PEG-8 caprylate, PEG-8 caprylate/captrate, PEG cocoates, PEG-15 dicocoate, PEG-2 diisononanoate, PEG-8 diisostearate, PEG dilaurates, PEG-dioleates PEG-distearates, PEG Ditallates, PEG-isostearates, PEG-jojoba acids, PEG-laurates, PEG linolenates, PEG myristates, PEG-oleates, PEG-palmitates, PEG-ricinoleates, PEG-stearates, PEG-tallates, and the like. Preferred is PEG-8 distearate.

Also suitable as emulsifiers are ethers and ether derivatives including dicapryl ether, batyl alcohol, batyl stearate, butoxyethanol, butoxypropanol, diethylene glycol, dimethyl ether, and so on.

Preferably the emulsifiers are a mixture of alkoxylated alcohols and alkoxylated carboxylic acids, in a ratio of 1:20 to 20:1, in combination with an ether, preferably dicapryl ether.

### Sunscreen Agents

It may also be desired to add various sunscreen agents in the stick composition of the invention. Examples of such sunscreen agents include p-aminobenzoic acid (PABA), cinoxate, diethanolamine p-methoxycinnamate, digalloyl trioleate, benzophenone 8, ethyl dihydroxypropyl PABA, octocrylene, octyl methoxycinnamate, octyl salicylate, glyceryl PABA, homosalate, methyl anthranilate, benzophenone 3, padimate A, padimate O, and so on. If sunscreen agents are included, a level of 0.001 to about 10, preferably 0.01-7, most preferably 0.01-5% is suggested.

### Pigments and Powders

If the stick compositions of the invention are in the form of color cosmetics, it may be desired to add 0.1-75%, preferably 1-50%, more preferably 5-45% dry particulate matter. The dry particulate matter may be pigments or noncolored or white powder materials having a particle size of 0.5-150, preferably 1-100 microns.

Preferred pigments include organic and inorganic pigments. Suitable inorganic pigments include the iron oxides such are red, yellow, blue, green, and the like. Suitable organic pigments include the D&C and FD&C reds, yellows, oranges, greens, blues, and the like, as well as the Lakes of D&C and FD&C colors.

The colored or white particulate materials include nylon, teflon, silica, titanium dioxide, chalk, alumina, calcium dioxide, kaolin, mica, talc, and so on.

The invention will be further described in connection with the following examples which are set forth for the purpose of illustration only.

### Example 1

An antiperspirant stick composition was made as follows:

| | w/w % |
|---|---|
| Aluminum zirconium tetrachlorohydrex gly | 25.00 |
| Isoeicosane | 30.00 |
| Diisopropyl sebacate | 30.00 |
| Paraffin | 10.00 |
| Polyolefin Elastomer* | 4.00 |
| Steareth-100 | 0.50 |
| PEG-8 distearate | 0.50 |

*ENGAGE POE, Dow Chemical Company

The polyolefine elastomer was heated to 150° C. to melt. The isoeicosane was added and the mixture was cooled to 100° C. and the paraffin added. The remaining ingredients were added and the composition was poured into sticks and allowed to cool.

### EXAMPLE 2

Two antiperspirant stick formulas were made as follows:

| | w/w % | |
|---|---|---|
| | A | B |
| Aluminum zirconium tetrachlorohydrex gly | 25.00 | 25.00 |
| Isododecane | 18.00 | 18.00 |
| Paraffin | 11.00 | 1.00 |
| Isoeicosane | 15.00 | 10.00 |
| Polyolefin elastomer* | 2.50 | 2.50 |
| PEG-8-distearate | 3.00 | 3.00 |
| Polyethylene | 5.00 | 5.00 |
| Diisopropyl sebacate | 20.00 | 20.00 |
| Fragrance | 0.50 | 0.50 |
| Dicapryl ether | — | 11.00 |
| Microcrystalline wax | — | 9.50 |

The compositions were mixed in the same manner as set forth in the previous example.

### EXAMPLE 3

A deodorant stick is prepared according to the following formula:

| | w/w % |
|---|---|
| Isododecane | 18.00 |
| Paraffin | 4.00 |
| Isoeicosane | 16.00 |
| Engage POE | 2.50 |
| PEG-8 distearate | 3.00 |
| Polethylene | 6.00 |
| Diisopropyl sebacate | 20.50 |
| Fragrance | 5.50 |
| Dicapryl ether | 12.00 |
| Microcrystalline wax | 12.50 |

### EXAMPLE 4

An analgesic stick is prepared according to the following formula:

| | w/w % |
|---|---|
| Isododecane | 18.00 |
| Paraffin | 4.00 |
| Isoeicosane | 16.00 |
| Engage POE | 2.50 |
| PEG-8 distearate | 3.00 |
| Polyethylene | 6.00 |
| Fragrance | 0.50 |
| Dicapryl ether | 12.00 |
| Microcrystalline wax | 12.50 |
| Methyl salicylate | 25.50 |

### EXAMPLE 5

A sunscreen stick having an SPF of 15 is prepared according to the following formula:

| | w/w % |
|---|---|
| Isododecane | 18.00 |
| Paraffin | 4.00 |
| Isoeicosane | 16.00 |
| Engage POE | 2.50 |
| PEG-8 distearate | 3.00 |
| Polyethylene | 6.00 |
| Diisopropyl sebacate | 20.42 |
| Fragrance | 0.50 |
| Dicapryl ether | 12.50 |
| Benzophenone 3 | 5.00 |

We claim:

1. A cosmetic stick composition comprising, by weight of the total composition:
0.1 -10% of a polyolefin elastomer.
10-70% of a solubilizer for the polyolefin elastomer, and
5-60% of a wax-type solidifying agent.

2. The composition of claim 1 wherein the polyolefin elastomer has a density of 0.85 to 0.97 grams per cubic centimeter.

3. The composition of claim 2 wherein wherein the polyolefin elastomer is a copolymer of ethylene with a C₃₋₂₀ alpha-olefin.

4. The composition of claim 3 wherein the polyolefin elastomer is of the formula: 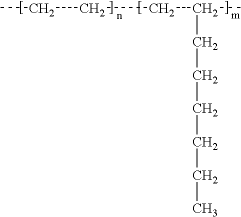
wherein m and n are each independently 1-1,000,000.

5. The composition of claim 1 wherein the solubilizer for the polyolefin elastomer is a liquid at room temperature.

6. The composition of claim 5 wherein the solubilizer for the polyolefin elastomer is a non-volatile oil.

7. The composition of claim 6 wherein the non-volatile oil is a paraffinic hydrocarbon.

8. The composition of claim 7 wherein the paraffinic hydrocarbon is a straight or branched chain paraffinic hydrocarbon having 5 to 50 carbons in the aliphatic chain.

9. The composition of claim 8 wherein the paraffinic hydrocarbon is a branched chain aliphatic hydrocarbon having 5 to 50 carbon atoms in the aliphatic chain.

10. The composition of claim 9 wherein the paraffinic hydrocarbon is isocicosane.

11. The composition of claim 1 wherein the wax-type solidifying agent has a melting point of 35 to 120° C.

12. The composition of claim 11 wherein the wax-type solidifying agent is is selected from the group consisting of a low melting point wax, a high melting point wax, and mixtures thereof.

13. The composition of claim 12 wherein the wax-type solidifying agent is a high melting point wax.

14. The composition of claim 13 wherein the wax-type solidifying agent is paraffin, microcrystalline wax, polyethylene, or mixtures thereof.

15. The composition of claim 1 additionally comprising 0.1-30% of an antiperspirant active.

16. The composition of claim 1 additionally comprising 0.1-30% of a deodorant active.

17. The composition of claim 1 additionally comprising 1-60% of an emollient oil.

18. The composition of claim 17 wherein the emollient oil is an ester.

19. The composition of claim 18 wherein the emollient oil is the reaction product of isopropyl alcohol and sebacic acid.

20. The composition of claim 1 further comprising 0.1-5% of a emulsifier.

21. The composition of claim 20 wherein the emulsifier is an alkoxylated alcohol or an alkoxylated carboxylic acid.

22. An anhydrous suspensoid antiperspirant stick composition comprising, by weight of the total composition:
0.1-10% of a polyolefin elastomer.
10-70% of a solubilizer for the polyolefin elastomer,
5-60% of a wax-type solidifying agent, and
1-30% antiperspirant active.

23. The composition of claim 22 wherein the polyolefin elastomer has a density of 0.85 to 0.97 grams per cubic centimeter.

24. The composition of claim 23 wherein the polyolefin elastomer is a copolymer of ethylene and a C₃₋₂₀ alpha-olefin.

25. The composition of claim 22 further comprising 1-60% of an emollient oil.

26. The composition of claim 25 further comprising 0.001-5% of an emulsifier.

27. The composition of claim 26 wherein the emulsifier is an alkoxylated alcohol, an alkoxylated carboxylic acid, or mixtures thereof.
